(19) United States
(12) Patent Application Publication
(10) Pub. No.: US 2022/0192939 A1
(43) Pub. Date: Jun. 23, 2022

(54) Composition For The Remineralization Of Teeth

(71) Applicant: Ivoclar Vivadent AG, Schaan (LI)

(72) Inventors: Urs Lendenmann, Grabs (CH); Carlo Bolis-Truog, Igis (CH); Michelle Alge, Lustenau (AT)

(21) Appl. No.: 17/549,959

(22) Filed: Dec. 14, 2021

(30) Foreign Application Priority Data
Dec. 18, 2020 (EP) 20215304.5

(51) Int. Cl. A61K 8/21; A61Q 11/00; A61K 8/49; A61K 8/44

(52) U.S. Cl. CPC A61K 8/21; A61Q 11/00; A61K 2800/30; A61K 8/44; A61K 8/4946

(57) ABSTRACT

A composition for use in a method for the therapeutic treatment of teeth, which contains at least one fluoride-containing component, at least one amino acid and a liquid carrier. The composition preferably has a pH of from 4 to 7 and preferably does not contain any abrasive or polymeric constituents.

### CROSS-REFERENCE TO RELATED APPLICATIONS

This application claims priority to European Patent Application No. 20215304.5 filed on Dec. 18, 2020, the disclosure of which is incorporated herein by reference in its entirety.

### TECHNICAL FIELD

The present invention relates to a composition for the remineralization of teeth.

### BACKGROUND

Caries is a disease of hard tooth tissue (enamel and dentine). It develops when acid removes minerals from the inorganic portion of the hard tooth tissue, which consists predominantly of hydroxyapatite. This reduces the density of the mineral and increases the permeability of the tooth structure to liquids and ions. The acid can directly reach the teeth through nutrition, above all through acidic drinks. Of greater importance, however, are carbohydrates, above all saccharose, which are fermented from biofilms on the teeth to form organic acids, above all lactic acid. The pH in the biofilm on the teeth can be lowered to approx. 4 to 5 within a few minutes (Lingstrom et al., 1993, J Dent Res, 72:865-870). If there is an undersaturation in relation to the solubility of hydroxyapatite in the saliva, then decalcifications form first as caries precursors (initial caries). These are recognizable macroscopically as white spots (Arends & Christoffersen, 1986, J Dent Res, 65:2-11). Unless the initial caries lesion has advanced so far that the surface collapses and a hole forms, it can be remineralized.

Although the possibility of remineralizing enamel test pieces was already proven in the laboratory before 1970 (Johansson, 1965, J Dent Res, 44:64-70; Feagin et al., 1969, Arch Oral Biol, 14:1407-1417), as yet there are no products which remineralize better than fluorides in a clinically detectable and predictable manner. In fact, however, it was already described many years ago that in vivo remineralization proceeds much more slowly than in the laboratory (Gelhard & Arends, 1984, J Biol Buccale, 12:49-57).

Until now, in the prevention of caries, above all good oral hygiene and the topical application of fluoride to the teeth have proved to be successful. Fluoride reduces the solubility of hydroxyapatite. Regular application of fluoride to teeth also leads to remineralization (Gao et al. 2016, BMC Oral Health, 16:12). However, this requires a great deal of time and success is difficult to predict, with the result that the application of fluoride, e.g. in the form of toothpastes or mouthwash solutions, only has a moderate effect.

Toothpastes additionally contain abrasives and thickening agents. The abrasives are used for the removal of dental plaque, while the thickening agents contribute to increasing the viscosity, in order thus to obtain a paste. Regular use of abrasive toothpastes can, above all in the case of poor cleaning technique, lead to increased enamel erosion and injuries to the gums. The fluoridating effect of toothpastes is only small. In addition, the remineralizing effect of toothpastes and mouthwash solutions is heavily dependent on the co-operation and the discipline of the user, since these products must be used repeatedly daily.

All this has a disadvantageous effect on the success of the remineralization. There is therefore a need for simple, rapid methods for the remineralization of teeth, which bring about a substantial remineralization in a short time in one step and after only a single treatment.

EP 3 513 777 A1 and corresponding US 20190224082, which US publication is hereby incorporated by reference in its entirety, disclose a method for the remineralization of teeth, in which a solution of a fluoride component is first applied to the teeth, followed by a sol or a colloid of a nano-calcium component. It is important that the fluoride component is applied to the tooth before the calcium component.

DE 10 2010 003 280 A1 discloses oral and dental care products which contain at least one oligo- or polypeptide of vegetable origin, in which the molar ratio of basic amino acids (arginine, histidine, lysine) to acidic and semi-acidic amino acids (aspartic acid, glutamic acid, tyrosine, cysteine) is greater than 1. The peptides are to promote the growth of microbes which are advantageous for oral health. In addition, they are to improve the remineralization of teeth through calcium-containing substances.

DE 38 16 237 A1 and corresponding U.S. Pat. No. 5,135,396, which US patent is hereby incorporated by reference in its entirety, disclose fine filling materials for dental purposes, which contain hydroxyapatite and preferably also a calcification-promoting protein. The protein is preferably selected from phosvitin, casein and histidine-rich proteins. The fine filler is to be suitable for repairing pits, cracks and surface lesions in the dental enamel.

### SUMMARY

The object of the invention is to provide compositions which are suitable for the remineralization of teeth, for the treatment of initial caries lesions, for caries protection and for the prevention and treatment of dental erosion. The compositions are to make the simple and rapid remineralization of teeth possible through a single treatment.

This object is achieved according to the invention by compositions which contain, in addition to a liquid carrier and at least one fluoride-containing component, at least one amino acid. For the remineralization of teeth, the composition is applied to the tooth areas to be treated. It was surprisingly found that the compositions according to the invention bring about a much better remineralization than known fluoride-containing preparations. Thus, the compositions according to the invention not only make efficient caries protection possible, that is to say they not only prevent lesions from forming or increasing in size, but also allow an effective treatment of initial caries lesions and the prevention and treatment of dental erosion.

### BRIEF DESCRIPTION OF THE DRAWINGS

The invention is explained in more detail below by the figures which show the following:

FIG. 1 shows a hardness profile of a healthy bovine tooth;

FIG. 2 shows a hardness profile of a bovine tooth after storage in a demineralization solution;

FIG. 3 shows hardness profiles of a bovine tooth which has been treated with an ammonium fluoride solution;

FIG. 4 shows the hardness profiles of a control test with an untreated initial lesion;

FIG. 5 shows the hardness profiles of teeth which have been treated with a composition according to the invention;

FIG. 6 shows the hardness profiles of teeth which have been treated with a composition according to the invention; and

FIG. 7 shows the hardness profiles of teeth which have been treated with a composition according to the invention.

### DETAILED DESCRIPTION

The invention relates to a composition and a method for the remineralization of teeth, in particular for the treatment of initial caries lesions, for example of fissures, smooth surfaces, interdental surfaces and dental crevices, for caries protection on selected tooth surfaces, and for the prevention and treatment of dental erosions. By dental erosion is meant the progressive loss of mineralized tooth tissue through chemical processes which are not caused by microorganisms.

The compositions according to the invention are characterized by the fact that they contain a fluoride component in combination with an amino acid. It was surprisingly found that the compositions according to the invention already clearly increase the hardness of teeth after a single, brief treatment.

Preferred fluoride components are sodium fluoride, potassium fluoride, rubidium fluoride, caesium fluoride, potassium bifluoride (KHF₂), silver(I) fluoride (AgF), tin(II) fluoride (SnF₂), ammonium fluoride, ammonium bifluoride, salts of fluorophosphoric acid, such as sodium monofluorophosphate, potassium monofluorophosphate and ammonium hexafluorophosphate, tetra-n-butylammonium dihydrogen trifluoride (TBAF-3), olaflur (INN), dectaflur (INN) or a mixture thereof. Particularly preferred fluoride compounds are ammonium fluoride, ammonium bifluoride, sodium fluoride, potassium fluoride, tetra-n-butylammonium dihydrogen trifluoride and mixtures thereof. Ammonium bifluoride, tetra-n-butylammonium dihydrogen trifluoride, olaflur (INN), dectaflur (INN), and in particular ammonium fluoride, are quite particularly preferred.

Compositions which contain more than 1500 ppm, preferably more than 5000 ppm, fluoride (relative to the fluoride anion) in dissolved form are preferred. The maximum concentration depends on the choice of the solvent and the fluoride. It preferably lies below 20 wt.-%, particularly preferably below 10 wt.-%. The compositions according to the invention preferably contain 0.15 wt.-% to 20 wt.-%, particularly preferably 0.5 wt.-% to 10 wt.-%, quite particularly preferably 1 to 10 wt.-% and most preferably 1 to 5 wt.-% fluoride ions. Unless otherwise indicated, all percentages herein relate to the total mass of the composition.

Non-naturally occurring amino acids, or preferably naturally occurring, particularly preferably proteinogenic amino acids, can be used as amino acids. Basic, neutral or acidic amino acids can be used according to the invention. Lysine, histidine, arginine, glutamine, asparagine, tyrosine, glycine, serine, cysteine, threonine, alanine, valine, methionine, leucine, isoleucine, proline, tryptophan, phenylalanine, glutamic acid, aspartic acid, hydroxyproline and mixtures thereof are preferred. Glutamine, asparagine, tyrosine, glycine, serine, cysteine, threonine, alanine, valine, methionine, leucine, isoleucine, proline, tryptophan, phenylalanine, glutamic acid, aspartic acid, hydroxyproline and mixtures thereof are particularly preferred. Histidine, and in particular phenylalanine and lysine, as well as mixtures thereof are quite particularly preferred amino acids. The amino acids are present in monomeric form. The combination of histidine and ammonium fluoride is particularly preferred according to the invention.

The amino acids are preferably used in a total quantity of from 0.01 to 0.5 mol/l, preferably 0.025 to 0.2 mol/l, relative to the total volume of the composition.

The compositions according to the invention also contain a liquid carrier. Solvents and solvent mixtures which can dissolve the fluoride compound(s), amino acid(s) and optionally present auxiliaries, such as for example substances for adjusting the pH, are preferred as carrier. According to the invention, those compositions in which all components are present in completely dissolved form are particularly preferred.

Preferred solvents are water, organic solvents miscible with water and mixtures thereof. Preferred organic solvents are ethanol, isopropanol, acetone, methanol and propylene glycol. Mixtures of water with at least one organic solvent are particularly preferred. Water, ethanol, isopropanol, acetone and mixtures thereof, in particular mixtures which contain water and at least one of the named organic solvents, are quite particularly preferred, and exclusively water is most preferably used.

Organic solvents can reduce the solubility of fluoride salts and amino acids. On the other hand, however, they are advantageous because they improve the wetting of the tooth tissue and/or accelerate the drying of the solution after the application. For applications in which e.g. a rapid drying is desired, organic solvents and solvent mixtures which contain at least one organic solvent are therefore preferred. An organic solvent or a solvent mixture, in which the fluoride component(s) and amino acid(s) used are completely soluble in the desired quantities, is preferably used. According to the invention, solvent mixtures are preferred which contain at most 25 vol.-% organic solvent and preferably more than 75 vol.-% water, in each case relative to the total volume of solvents.

The compositions according to the invention preferably have a pH of from 3 to 8, particularly preferably from 4 to 7, quite particularly preferably 4 to 6, and most preferably from 4.5 to 5.5. The pH can optionally be adjusted by adding acid(s) or base(s). The acid(s) or base(s) can be inorganic or organic. Acids preferred according to the invention are hydrochloric acid, phosphoric acid, nitric acid, sulfuric acid, lactic acid, acetic acid, formic acid, citric acid and mixtures thereof. The most preferred acid is hydrochloric acid. Bases preferred according to the invention are sodium hydroxide, potassium hydroxide, ammonium hydroxide, 2-aminomethyl propanol, tris-(hydroxymethyl)-aminomethane, tetrahydroxypropyl ethylenediamine and mixtures thereof.

According to the invention, those compositions which do not contain any calcium salts and/or any phosphates and in particular any calcium phosphate are particularly preferred. In addition, according to the invention, those compositions which do not contain any abrasive and/or polymeric components are preferred. By phosphates is meant here the salts of orthophosphoric acid. This does not include the salts of fluorophosphoric acid, such as e.g. sodium monofluorophosphate and potassium monofluorophosphate.

In particular, the compositions according to the invention preferably do not contain any di- or monohydrogen phosphates such as monocalcium phosphate Ca(H₂PO₄)₂, dicalcium phosphate CaHPO₄, tricalcium phosphate Ca₃(PO₄)₂, octacalcium phosphate Ca₈H₂(PO₄)₆, any amorphous calcium phosphate (ACP), any di- or polyphosphates such as dicalcium diphosphate Ca₂P₂O₇ or calcium triphosphate Ca₅(P₃O₁₀)₂, any hydroxy- or oxophosphates, such as hydroxyapatite Ca₅(PO₄)₃(OH), apatite Ca₁₀(PO₄)₆(OH, F, CI, Br)₂, or tetracalcium phosphate Ca₄(PO₄)₂₀, either in anhydrous form or in the form of the hydrates. In addition, the compositions according to the invention preferably do not contain any calcium carbonate CaCO₃, calcium chloride CaCl₂, calcium fluoride CaF₂, calcium glycerophosphate, calcium hydroxide Ca(OH)₂, calcium sulfate CaSO₄, calcium lactate or calcium gluconate, either in anhydrous form or in the form of the hydrates.

The compositions according to the invention for use in the remineralization of teeth preferably contain:
(a) 50 to 99.5 wt.-%, preferably 75 to 99 wt.-%, particularly preferably 85 to 98 wt.-% and quite particularly preferably 86 to 96 wt.-% liquid carrier, preferably water,
(b) 0.15 wt.-% to 20 wt.-%, preferably 0.5 wt.-% to 10 wt.-% and particularly preferably 1 to 5 wt.-% (relative to F⁻) at least one fluoride-containing component, preferably ammonium fluoride, in each case relative to the total weight of the composition,
(c) 0.01 to 0.5 mol/l, preferably 0.025 to 0.2 mol/l and particularly preferably 0.05 to 0.1 mol/l at least one amino acid, preferably histidine, relative to the total volume of the composition and
(d) optionally means for adjusting the pH, preferably hydrochloric acid.

A particularly preferred composition comprises:
(a) water in the amount required to reach 100%,
(b) 5 wt.-% (relative to F⁻) of ammonium fluoride, in each case relative to the total weight of the composition,
(c) 0.1 mol/l of histidine, relative to the total volume of the composition and
(d) hydrochloric acid to adjust the pH to 5.

In addition to the named components, the compositions according to the invention can advantageously contain further additives (e). Preferred additives are butyl diglycol, sweetening agents, flavouring agents and surfactants. Surfactants promote the dissolution of the pellicle and/or deposits usually present on the tooth surface. Additives are optionally added in a total quantity of from 0.01 to 5 wt.-%, preferably 0.1 to 3 wt.-% and particularly preferably 0.5 to 2 wt.-%, relative to the total weight of the composition.

Those compositions in which the individual constituents are selected from the above-defined preferred and particularly preferred substances are naturally particularly preferred according to the invention. Compositions which consist exclusively of the named constituents are quite particularly preferred.

The compositions according to the invention preferably do not contain any thickening agents, any humectants or any gelling agents, in particular any sorbitol and/or glycerol.

Method For The Remineralization Of Teeth

For the remineralization of one or more teeth, the composition according to the invention is applied to the tooth surface to be treated. The method according to the invention preferably comprises the following steps:

(i) optional pre-treatment of the tooth or teeth,

(ii) optional etching of the tooth or teeth,

(iii) application of a composition according to the invention and

(iv) optional application of a composition comprising a particulate calcium salt.

Optional Pre-Treatment Of The Tooth Or Teeth

The tooth areas to be treated are preferably cleaned before the actual treatment. In the process tartar (calculus) and other deposits on the teeth are removed. A professional dental cleaning is particularly preferably carried out.

As a thin film of debris, the so-called smear layer, can remain on the tooth after a (professional) dental cleaning completed by a polishing, it is preferred to etch the surface of the tooth areas to be treated after the cleaning so that the composition according to the invention can penetrate deep into the dental enamel. For this, dots of an etchant are applied to the regions in question, in particular the demineralized initial lesions visible as white spots, and left there for a short time. The etchant is then rinsed off. The reaction time of the acid is not very critical. It should be long enough to dissolve the smear layer, but not so long that the dental enamel is unnecessarily weakened. Times of between 1 s and 1000 seconds, preferably 5 s to 120 s, particularly preferably 5 s to 60 s, are preferred according to the invention.

Aqueous solutions of phosphoric acid, hydrochloric acid, nitric acid, sulfuric acid, lactic acid, acetic acid, formic acid, citric acid and ethylenediaminetetraacetic acid come into consideration in particular as acids for the etching. Phosphoric acid is particularly preferred, preferably 37% phosphoric acid.

So that these acid solutions can be applied in dots precisely, they preferably contain a thickener. Suitable etchants are known per se from dental adhesive technology and have been thoroughly described. 37% phosphoric acid etching gel, such as e.g. the commercially available gel called Total Etch from Ivoclar Vivadent AG, is preferred.

The etchant is rinsed off with water after the desired reaction time and the tooth is then dried, preferably with a stream of air, but other methods are also suitable.

Application Of The Composition According To The Invention

After the cleaning and/or the acid treatment of the tooth surface, the composition according to the invention is applied to the tooth to be treated or preferably to the entire natural set of teeth, e.g. with a small brush, sponge or a syringe with a blunt needle. As a rule, approximately 0.5 ml or less, preferably 0.1 to 0.3 ml, of the composition according to the invention is required to treat the entire set of teeth. Alternatively, the composition can be applied in a targeted manner to the sites to be treated, particularly when no professional dental cleaning has been carried out beforehand. The reaction time after the application of the composition is preferably in each case less than 5 minutes, particularly preferably 0.5 to 2 minutes and quite particularly preferably 0.5 to 1 minute. After the reaction time of the composition according to the invention has expired, the teeth can optionally be dried, e.g. with an air syringe. Any possible excess need not be removed. An immediately following second application of the composition is possible, but as a rule not necessary, in order to achieve the desired effect. A repeated, i.e. second or third, application is advantageous in particular when not all sites to be treated were reached by the composition during the first application.

It was surprisingly found that the compositions according to the invention already achieve a high effect after a single application. It was particularly surprising that this effect is already achieved after a short reaction time. As indicated above, according to a particularly preferred embodiment this is only 0.5 to 2 minutes, wherein a reaction time of 1 minute is quite particularly preferred. This is a substantial advantage with regard to use by the dentist because the treatment is already completed after a short time, which is much easier both for the patient and for the staff carrying out the treatment.

The compositions according to the invention are particularly suitable for therapeutic use by the dentist, in particular for the remineralization of teeth, for caries protection and quite particularly for the therapeutic treatment and prevention of initial caries lesions (initial caries), of white spots, of dental erosion, for hardening damaged teeth and for the restoration of hard tooth tissue. The compositions not only bring about a clear increase in the hardness of the upper tooth layers but also a lasting protection of the teeth against subsequent acid attacks. This effect is achieved through the use of the composition according to the invention alone.

Therefore, no further treatment steps with therapeutically active agents are preferably carried out. Non-therapeutic measures, such as e.g. rinsing the teeth after the application, are not ruled out.

A preferred embodiment of the invention are thus agents for use in a method for the therapeutic treatment of teeth, i.e. for caries protection, for the treatment and prevention of initial caries lesions (initial caries), of white spots, of dental erosion, for hardening damaged teeth and for the restoration of hard tooth tissue, wherein the composition is applied to the tooth or teeth one to three times, preferably once, and in each case left to react for a period of less than 5 minutes, preferably 0.5 to 2 minutes, particularly preferably 0.5 to 1 minute and quite particularly preferably 1 minute.

According to a particularly preferred embodiment of the invention, a composition comprising a particulate calcium salt is applied to the tooth surface after the application of the composition comprising one or more amino acids and a fluoride source. In the following, the calcium salt-comprising composition is referred to as calcium component.

The calcium component contains a calcium salt, preferably calcium fluoride, calcium carbonate, calcium sulphate, calcium silicate, calcium oxide or calcium hydroxide. Calcium fluoride, calcium carbonate, calcium sulphate and calcium silicate, quite particularly calcium carbonate and in particular calcium fluoride are particularly preferred. According to the invention the calcium salt is present in nanoparticulate form. Nanoparticles with a particle size of <100 nm, particularly preferably <40 nm and quite particularly preferably <30 nm, are preferred. The particle size preferably lies in a range of from 1 to 100 nm, particularly preferably 1 to 40 nm and quite particularly preferably 1 to 30 nm.

In all cases, unless expressly stated otherwise, the particle size is the average particle diameter (number average) measured using dynamic light scattering (DLS), preferably using a Malvern ZetaSizer. Nanoparticles having an average particle size within a range of 5 to 40 nm are most preferred.

The calcium component preferably contains no phosphate, in particular no calcium phosphate.

The nanoparticulate calcium salt is present as a sol in a volatile suspending agent. In particular orally acceptable solvents which evaporate on their own within a short time under normal ambient conditions or under oral conditions or can be dried up with the aid of an air jet come into consideration as suspending agents for the calcium particles. Preferred suspending agents are alcohols, esters, ethers, ketones, alkanes, alkenes, water or mixtures thereof, in particular ethanol, methanol, n-propanol, i-propanol, n-butanol, sec-butanol, isoamyl alcohol, acetone, water, acetonitrile, ethyl acetate, methoxy propanol, dibutyl ether, dioxane, methyl ethyl ketone, heptane, hexane or dimethylformamide. Ethanol, acetone, isopropanol, water and mixtures thereof are particularly preferred. Organic solvents or mixtures of organic solvents and water are preferred, anhydrous solvents are particularly preferred. The most preferred solvent is ethanol.

An important aspect of the invention is that the calcium component contains nanoparticulate particles of a calcium salt. The calcium salt and also the type and quantity of the suspending agent are therefore chosen such that the particles do not dissolve in the solvent. They have to be present in particulate form at least during application. The nanoparticulate calcium salt and the suspending agent can be present in separated form. In this case, the calcium salt is dispersed in the suspending agent before application. Ready-made suspensions (sols) of the calcium particle or calcium particles are preferred.

Suspensions which have a pH of less than 11.0 and preferably less than 10 are preferred according to the invention. In the case of anhydrous suspensions, the suspension is mixed with water in a ratio of 1:1 to determine the pH. The pH preferably lies in a range of from 4.4 to 11, particularly preferably 5 to 10 and quite particularly preferably 6 to 10.

The proportion of the calcium salt in the sol or colloid can lie between 0.0001 and 99.9999 wt.-%. Proportions of from 0.1 wt.-% to 40 wt.-%, particularly preferably 1 wt.-% to 30 wt.-%, quite particularly preferably 4 wt.-% to 25 wt.-% and in particular 5 wt.-% to 15 wt.-%, are preferred. However, the proportion should also not be too high, so that the sol or colloid remains thin enough to be able to be applied to the tooth using a brush or small brush.

A particularly preferred calcium component comprises 10±5 wt.-% calcium fluoride having an average particle size within a range of 5 to 40 nm, sodium lactate, optionally one or more further additives (e) as defined above and ethanol in the amount required to reach 100 wt.-%.

The calcium component can be applied over the whole surface of the entire set of teeth or only to the areas to be treated. After the sol has been applied to the tooth surface, the nanoparticles form a solid layer on the tooth surface. The layer formation is preferably effected by evaporation of the solvent, wherein the drying can be actively accelerated, for example by blowing the solvent with an air jet. After the drying, the dried layer can remain on the tooth. It is worn away in the course of time, for example during tooth brushing. However, it is also possible to actively remove the layer after the treatment.

According to the present invention, compositions, which comprises two separate components are particularly preferred, i.e. a first component comprising one or more amino acids and a fluoride source and a second component comprising a particulate calcium salt. The composition is designed for the successive application of the first component and the second component to the tooth surface, wherein the first component is applied to the tooth before the second component.

The invention is explained in more detail below by means of figures and examples.

FIG. 1 shows the hardness profile of a healthy bovine tooth. The hardness of the tooth at the surface (distance=0 μm) corresponds to the hardness in deeper regions (distance=300 μm).

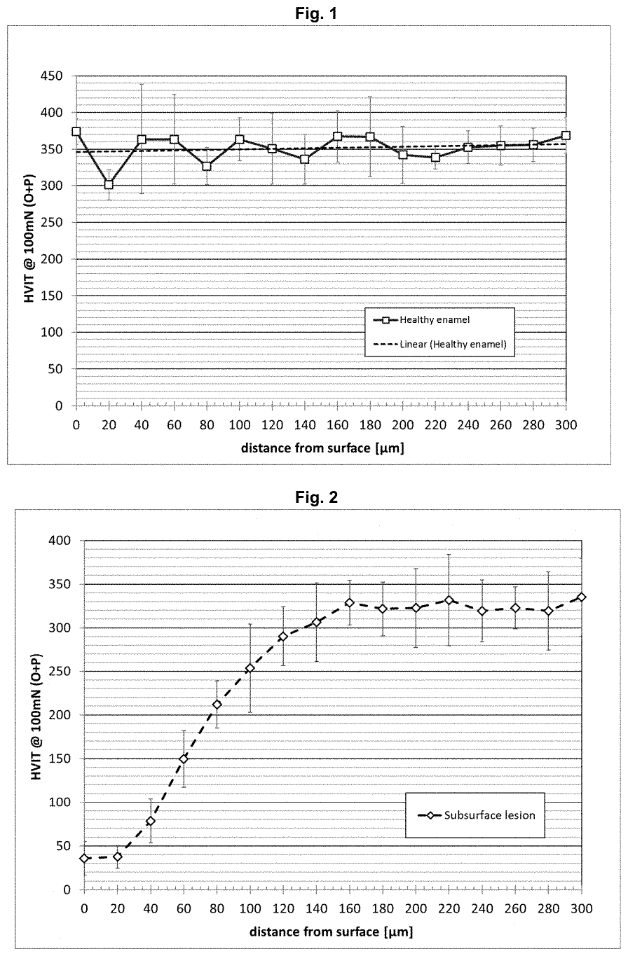

FIG. 2 shows the hardness profile of a bovine tooth after storage in a demineralization solution. Here, the Vickers hardness (HV_{IT}) at the tooth surface which was in direct contact with the demineralization solution is clearly reduced (from approx. 320 HVIT to approx. 40 HVIT). The hardness profile shows that the demineralization has an effect in the chosen conditions up to a depth of approx. 160 μm, measured from the tooth surface. From a depth of approx. 160 μm, the tooth has the natural hardness.

FIG. 3 shows the hardness profiles of a bovine tooth which has been treated with an ammonium fluoride solution (5% ammonium fluoride, without addition of amino acid) not according to the invention (Example 1). The dashed line shows the profile of the untreated enamel sample and the continuous line shows the hardness profile of the enamel sample which has been treated with the fluoride solution. The hardness profiles show that although the ammonium fluoride solution brings about a moderate remineralization in a narrow region close to the surface, in deeper regions a further demineralization is observed.

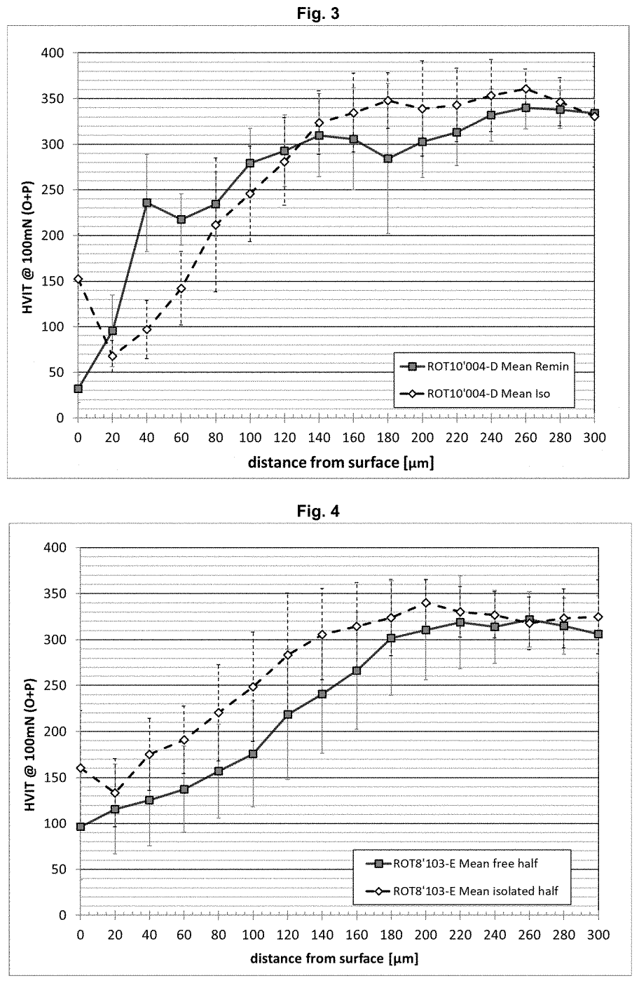

FIG. 4 shows the hardness profiles of a control test with an untreated initial lesion (negative control; Example 2). Half of the initial lesion was covered with nail varnish and the teeth were then stored in the demineralizing solution for a further 7 days. A further contact with the demineralizing solution is prevented by the nail varnish. The dashed line shows the profile of the half of the enamel samples isolated using nail varnish, the continuous line shows the hardness profile of the untreated enamel samples. It can be seen that the continued storage of the teeth in the demineralizing solution leads to a further decrease in the hardness. This means that the lesion becomes larger and the teeth are demineralized further.

FIG. 5 shows the hardness profiles of teeth which have been treated with a composition according to the invention (Example 3; 0.1M lysine and 5% ammonium fluoride; continuous curve), compared with an untreated enamel sample (dashed curve). The hardness profiles show that the treatment with the solution according to the invention brings about a clear increase in hardness of the teeth.

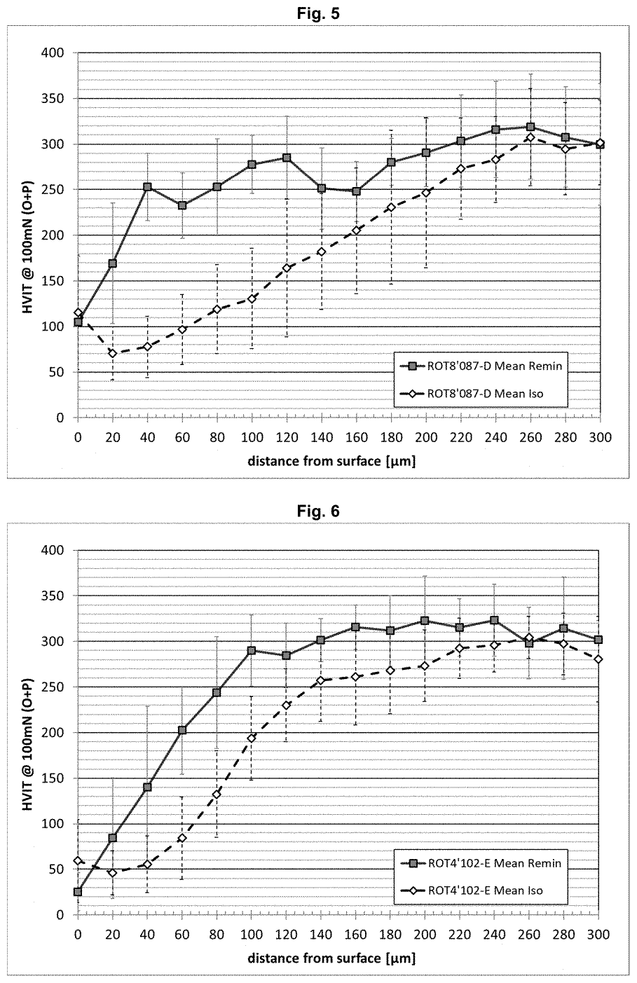

FIG. 6 likewise shows the hardness profiles of teeth which have been treated with a composition according to the invention (Example 4; 0.1M histidine and 5% ammonium fluoride; continuous curve), compared with an untreated enamel sample (dashed curve). The hardness profiles show that the treatment with the solution according to the invention brings about an increase in hardness in wide regions of the lesion.

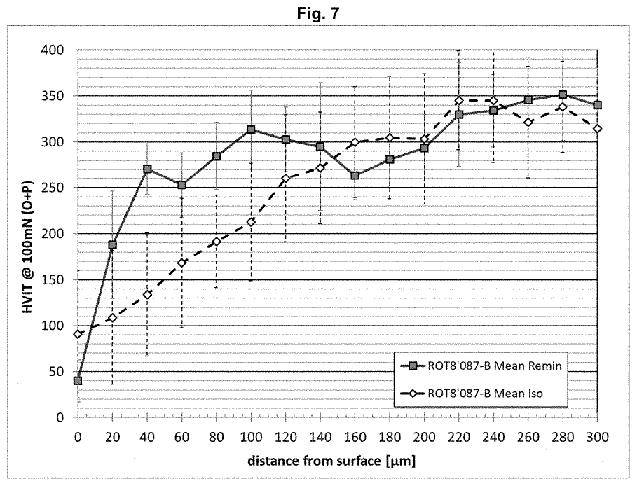

FIG. 7 shows the hardness profiles of teeth which have been treated with a composition according to the invention (Example 5; 0.1M phenylalanine and 5% ammonium fluoride; continuous curve), compared with an untreated enamel sample (dashed curve). The hardness profiles show that the treatment with the solution according to the invention brings about a clear increase in hardness, above all of the regions close to the surface.

### EXAMPLES

### Determination of the Remineralization Potential in a Caries-Producing Environment

The remineralization potential on the tooth was tested in a model with a chemically produced initial lesion in bovine enamel. For this, bovine teeth were embedded in resin, and the enamel was exposed and polished with SiC sandpaper, accompanied by water cooling. An artificial lesion was produced in the enamel by storage in a demineralizing solution for 14 to 21 days at 37° C. The demineralizing solution contained 50.0 mmol/l acetic acid, 3.0 mmol/l KH₂PO₄, 3.0 mmol/l CaCl₂·2 H₂O, 1.0 ppm methylenediphosphonic acid as well as 100 ppm sodium azide as preservative. The pH was adjusted to pH 5.0 with KOH.

The thus-produced lesions were half-covered with nail varnish, and the free surface was treated with the composition according to the invention. The test piece was then stored in the demineralizing solution at pH 5.0 at 37° C. for a further 7 days. Each treatment was carried out on three teeth each.

After the storage, the test pieces were rinsed briefly with water, patted dry and the nail varnish was removed from the isolated half with ethanol. The surface hardness of both halves was measured using a nanoindenter. For this, the surface of the test pieces was embedded with resin, in order then to be able to saw a disc out of the tooth cross section using a diamond saw. After the cross section had been polished, three hardness profiles each of the treated and isolated sides were measured (impressions with a distance of 20 μm in each case vertically up to a depth of 300 μm, Berkovich indenter, 100 mN load, loading with 400 or 600 mN/min., 2 s holding time at Fₘₐₓ). Hardness profiles were generated using the Vickers hardness values (HV_{IT}) calculated automatically by the device.

### Example 1

### Remineralization with Ammonium Fluoride Solution (Comparison Example)

Remineralization of an initial lesion by application of a 5% ammonium fluoride solution in water (2.57 wt.-% fluoride; adjusted to pH 5.0) for 1 minute. After the solution had been applied, the teeth were stored in the demineralizing solution for 7 days. The hardness profiles of this sample are reproduced in FIG. 3. The dashed line shows the profile of the half of the enamel samples isolated using nail varnish. The continuous line shows the hardness profile of the enamel sample which has been treated with the ammonium fluoride solution (without the addition of amino acid). The hardness profiles in FIG. 3 show a decrease in hardness at the surface in both cases. Although the treatment with the ammonium fluoride solution brings about a moderate remineralization in a narrow region close to the surface, in deeper regions a further demineralization is observed.

### Example 2

Control Test with an Untreated Initial Lesion (Negative Control; Comparison Example)

The teeth half-covered with nail varnish were stored in the demineralizing solution for a further 7 days. The side not covered with nail varnish was not treated. The dashed line shows the profile of the half of the enamel samples isolated using nail varnish, the continuous line shows the hardness profile of the untreated enamel samples. The hardness profiles in FIG. 4 show that the continued storage in the demineralizing solution without treatment leads to a further decrease in the hardness. This means that the lesion becomes larger, thus demineralization takes place.

### Example 3

### Remineralization with a Lysine-Containing Ammonium Fluoride Solution

Remineralization of an initial lesion by application of a 0.1M lysine solution with 5% ammonium fluoride in water (2.57 wt.-% fluoride; adjusted to pH 5.0) for 1 minute. After the solution had been applied, the teeth were stored in the demineralizing solution for 7 days. The hardness profiles in FIG. 5 show that the treatment with the solution according to the invention brings about a clear increase in hardness (continuous line).

### Example 4

### Remineralization with a Histidine-Containing Ammonium Fluoride Solution

Remineralization of an initial lesion by application of a 0.1M histidine solution with 5% ammonium fluoride in water (2.57 wt.-% fluoride; adjusted to pH 5.0) for 1 minute. After the solution had been applied, the teeth were stored in the demineralizing solution for 7 days. The hardness profiles in FIG. 6 show that the treatment with the solution according to the invention brings about an increase in hardness in wide regions of the lesion (continuous line).

### Example 5

### Remineralization with a Phenylalanine-Containing Ammonium Fluoride Solution

Remineralization of an initial lesion by application of a 0.1M phenylalanine solution with 5% ammonium fluoride in water (2.57 wt.-% fluoride; adjusted to pH 5.0) for 1 minute. After the solution had been applied, the teeth were stored in the demineralizing solution for 7 days. The hardness profiles in FIG. 7 show that the treatment with the solution according to the invention brings about a clear increase in hardness, above all in the regions close to the surface (continuous line).

1. A composition for use in a method for the remineralization of teeth, which comprises
(a) a liquid carrier,
(b) at least one fluoride-containing component and
(c) at least one amino acid.

2. The composition according to claim 1 wherein the remineralization of teeth comprises any of the following: caries protection, therapeutic treatment and prevention of initial caries lesions (initial caries), therapeutic treatment and prevention of white spots, therapeutic treatment and prevention of dental erosion, hardening damaged teeth, and restoration of hard tooth tissue.

3. The composition according to claim 1, wherein the composition is applied to the tooth or teeth one to three times, and in each application, left to react for a period of less than 5 minutes.

4. The composition according to claim 1, which does not contain any abrasive and/or any polymeric constituents.

5. The composition according to claim 1, which has a pH of from 4 to 7.

6. The composition according to claim 1, wherein the fluoride-containing component comprises sodium fluoride, potassium fluoride, ammonium fluoride, ammonium bifluoride, sodium monofluorophosphate, potassium monofluorophosphate, tetra-n-butylammonium dihydrogen trifluoride, rubidium fluoride, caesium fluoride, potassium bifluoride (KHF₂), silver(I) fluoride (AgF), tin(II) fluoride (SnF₂), olaflur, dectaflur or a mixture thereof.

7. The composition according to claim 1, which comprises more than 1500 ppm fluoride (F⁻), relative to the total mass of the composition.

8. The composition according to claim 1, wherein the amino acid comprises lysine, histidine, phenylalanine, arginine, glutamine, asparagine, tyrosine, glycine, serine, cysteine, threonine, alanine, valine, methionine, leucine, isoleucine, proline, tryptophan, glutamic acid, aspartic acid, hydroxyproline or a mixture thereof.

9. The composition according to claim 1, which comprises 0.01 to 0.5 mol/l amino acid, relative to the total volume of the composition.

10. The composition according to claim 1, which comprises, as fluoride-containing component (b), ammonium bifluoride, tetra-n-butylammonium dihydrogen trifluoride, olaflur (INN), dectaflur (INN), ammonium fluoride or a mixture thereof and/or, as amino acid (c), histidine, phenylalanine, lysine or a mixture thereof.

11. The composition according to claim 1, which comprises, as liquid carrier, water, ethanol, isopropanol, acetone, methanol, propylene glycol or a mixture thereof.

12. The composition according to claim 1, which comprises
(a) 50 to 99.5 wt.-% liquid carrier,
(b) 0.15 wt.-% to 20 wt.-% (relative to F⁻) at least one fluoride-containing component, relative to the total weight of the composition,
(c) 0.01 to 0.5 mol/l at least one amino acid, relative to the total volume of the composition.

13. The composition according to claim 12, which additionally comprises
(d) optionally means for adjusting the pH and/or
(e) 0.01 to 5 wt.-% further additives, relative to the total weight of the composition.

14. The composition according to claim 1, wherein the method comprises the following steps:
optional pre-treatment of the tooth or teeth,
(ii) optional etching of the tooth or teeth and
(iii) application of the composition.

15. A method of using the composition according to claim 1 for the remineralization of teeth, for caries protection, for the treatment and prevention of initial caries lesions (initial caries), of white spots, for the prevention or treatment of dental erosion, for hardening damaged teeth or for the restoration of hard tooth tissue comprising
applying the composition to a tooth or teeth one to three times, and
in each application, leaving the composition to react for a period of less than 5 minutes.
